(19) United States
(12) Patent Application Publication
(10) Pub. No.: US 2015/0379220 A1
(43) Pub. Date: Dec. 31, 2015

(54) DEVICE AND SYSTEM COMMUNICATING WITH A SUBJECT

(71) Applicant: ORIDION MEDICAL 1987 LTD., Jerusalem (IL)

(72) Inventors: Paul Stanley Addison, Edinburgh (GB); Michal Ronen, Givat-Brenner (IL); Konstantin Goulitski, Holon (IL)

(21) Appl. No.: 14/315,359

(22) Filed: Jun. 26, 2014

(51) Int. Cl. G06F 19/00; A61B 5/00; A61B 5/0476; A61B 5/0205; A61B 5/0402

(52) U.S. Cl. CPC G06F 19/3406; A61B 5/0205; A61B 5/0402; A61B 5/0476; A61B 5/741; A61B 5/742; A61B 5/083

(57) ABSTRACT

Provided are device, system and method for communicating with a subject, based on the condition of the subject, by providing instructions to the subject and/or to a health care provider.

### TECHNICAL FIELD

The present disclosure generally relates to medical monitoring systems and methods of using the same while communicating with a subject.

### BACKGROUND

Medical monitoring devices are routinely used in various medical settings to obtain or measure medical parameters relating to a patient's medical condition. The parameters and measurements are used to determine the health condition of the subject and allow monitoring the condition over time. In most instances, the health condition of the subject is being checked by a health care provider, which may then instruct the subject as needed, to perform procedures that may to improve the health condition.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided monitoring systems and methods of using the same, capable of interacting with a subject being monitored, for example, by providing instructions to the subject, whereby the instructions are related to the condition of the subject, or to changes to the condition of the subject. In some embodiments, the instructions may be used to prepare the subject to an intervention procedure or a physical examination. In some embodiments, the systems, devices and methods provided herein are bedside devices and systems capable of providing visual and/or audible instructions to a subject that is being monitored by one or more monitoring devices, wherein the instructions provided to the subject are based on one or more parameters obtained from the medical monitoring device. In some embodiments, the instructions may be provided to the health care provider. In some embodiments, the instructions are provided automatically and may be selected from a pre-determined set of instructions. In some embodiments, the instructions may be selected from, but not limited to: instructions for an intervention procedure to be taken by the subject, instructions for an intervention procedure to be taken by the health care provider; an indication to the subject, or combinations thereof.

In some embodiments, the systems, devices and methods provided herein take use of measurements or related information obtained from one or more medical monitoring devices. In some embodiments, the medical monitoring devices may be selected from, but not limited to: capnograph, pulse oximeter, heart monitor, blood pressure measurements, and the like. In some embodiments, the measurements or related information obtained from the medical monitoring devices are various physiological parameters, such as, for example, but not limited to: breath related parameters (as determined, for example, based on CO₂ measurements in exhaled breath), heart rate related parameters, blood related parameters, brain activity related parameters, and the like, or combinations thereof. According to some embodiments, the medical monitoring device are configured to interact with a communication device capable of providing instructions to a subject being monitored by the medical monitoring device, whereby the instructions are determined/issued/selected based on one or more parameters obtained from the medical monitoring device. In some embodiments, the communicating unit may include a display (monitor), a speaker, a tactile unit, each capable of interacting with the subject by providing instructions to the subject.

In some embodiments, the measured parameters may be further manipulated and/or processed prior to or simultaneously while being integrated by the processing unit (processing logic) of the communicating device.

Advantageously, the systems, devices and methods disclosed herein, allow automatic (or at least semi-automatic) interaction with a subject, by providing instructions to the patient, based on medical parameters being monitored in real time. Thus, the systems and methods disclosed herein advantageously provide an efficient, accurate, cost efficient and time saving means for interacting with a subject in real time by providing instructions related to the real time condition of the subject. Additionally, the systems and methods disclosed herein may provide feedback to the subject and/or to the health care provider, with respect to the condition of the subject and to the efficiency of intervention being performed by the subject himself and/or the health care provider, respectively. Further, the instructions may be provided by means that allow specific adaption to the subject, by selecting, for example, language of instructions, type of voice (i.e., male, female) providing instructions, volume of instructions; type and size of the displayed instructions, and the like.

According to some embodiments, there is provided a system for communicating with a subject, the system comprising: one or more medical monitoring devices configured to measure one or more physiological parameters of the subject; a communicating device configured to integrate the one or more physiological parameters of the subject to determine the condition of the subject, based on the measured parameters and provide instructions to the subject or to a health care provider, wherein the instructions are related to the condition of the subject.

According to some embodiments, the medical monitoring device may include a capnograph, a pulse oximeter, an Electrocardiogram (ECG), or combinations thereof.

According to some embodiments, the physiological parameters of the subject may include: breath related parameters, heart related parameters, blood related parameters, brain electrical activity, parameters derived therefrom, or combinations thereof.

According to some embodiments, the breath related parameters may include such parameters as, but not limited to: airflow, respiration rate, respiration effort, expired CO₂, or combinations thereof. Various expired CO₂ related parameters may include such parameters as, but not limited to: CO₂ concentration in breath, EtCO₂, CO₂ waveform, Respiration rate, respiration effort, data derived therefrom, and combinations thereof.

According to some embodiments, the heart related parameters may include heart rate, amplitude of cardiac pulses, Percent Modulation (PMod) of the cardiac pulses, or combinations thereof.

According to some embodiments, the blood related parameters may include blood pressure, SpO₂, or both.

According to some embodiments the instructions may include visual and/.or audible instructions. In some embodiments, the instructions may comprise instructions for an intervention procedure to be taken by the subject, instructions for an intervention procedure to be taken by the health care provider; an indication to the subject, or combinations thereof.

In some embodiments, the communicating device may include a display unit and/or an audio unit.

In some embodiments, the instructions may be selected from a predetermined set of instructions.

According to some embodiments, the display unit may be configured to display one or more of: one or more of the measured parameters, parameters derived from the measured parameters, an indication for an intervention to be taken by the subject, an indication for an intervention to be taken by a health care provider, an indication related to a health care provider, or combinations thereof.

According to some embodiments, the audio unit may include a speaker an earphone, and the like. In some embodiments, the audio unit is configured to provide audible instructions to the subject and/or to the health care provider.

According to some embodiments, the communicating device may further include a processing logic. In some embodiments, the processing logic may be configured to integrate the one or more physiological parameters of the subject to determine the condition of the subject.

According to some embodiments, there is provided a device for communicating with a subject, the device comprising: a) a processing logic configured to obtain and integrate one or more measured physiological parameters of the subject, obtained from one or more medical monitoring devices, to determine the condition of the subject, and b) a display unit and/or an audio unit configured to provide instructions to the subject and/or to a health care provider, wherein the instructions are related to the condition of the subject.

In some embodiments, the display unit may include a monitor. In some embodiments, the audio unit may include a speaker.

In some embodiments, the device may further include a user interface configured to control operating parameters of the device. The operating parameters may include volume (loudness), type of voice providing instructions, operating language, language of voice providing instructions, a recorder configured to pre-record instructions, or combinations thereof. In some embodiments, the user interface may be used by the subject and/or the health care provider to input or provide instructions and/or feedback to the device. For example, the user interface may be operated by the subject and/or health care provider by pressing a key, pressing a button, typing on a keyboard, talking to a microphone of the device, and the like. In some embodiments, the feedback/instructions that may be provided/input to the device may include such inputs as, but not limited to: confirm status, start a procedure, start operation of the device, end a procedure, end operation of the device, cancel a procedure, cancel operation of the device, confirm the instructions from the device are met by the subject and/or health care provider, and the like.

According to some embodiments, there is provided a method for communicating with a subject, the method comprising: a) obtaining a measurement of one or more physiological parameters of the subject; b) determining the condition of the subject based on the measured parameters of the subject; and c) issuing audible and/or visual instructions to the subject, wherein the instructions are related to the condition of the subject and are configured to alleviate said condition.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

FIG. 1—a schematic block diagram of a device communicating with a subject, according to some embodiments;

FIG. 2 schematic presentation of a system for communicating with a subject, according to some embodiments;

FIG. 3—a schematic block diagram of steps in a method for communicating with a subject, according to some embodiments; and

FIG. 4—a block diagram of an exemplary set of instructions provided by the device and system to a subject for performing a passive leg raising maneuver.

### DETAILED DESCRIPTION

In the following description, various aspects of the disclosure will be described. For the purpose of explanation, specific configurations and details are set forth in order to provide a thorough understanding of the disclosure. However, it will also be apparent to one skilled in the art that the disclosure may be practiced without specific details being presented herein. Furthermore, well-known features may be omitted or simplified in order not to obscure the disclosure.

As referred to herein, the terms “user”, “medical user”, “health care provider” and “health care professional” may interchangeably be used. The terms may include any health care provider who may treat and/or attend to a patient. A user may include, for example, a nurse, respiratory therapist, physician, anesthesiologist, and the like. In some cases, a user may also include a patient.

As referred to herein, the terms “monitoring device” and “medical device” may interchangeably be used. Exemplary monitoring devices include such devices as, but not limited to: Capnograph, pulse oximeter, Electrocardiograph (ECG), skin humidity measuring device, thermometer, Brain electrical activity monitoring device (such as BIS), and the like.

As referred to herein, the term “physiological parameter” is directed to a health related parameter of the subject. The health related parameter may be directly and/or indirectly measured, detected and/or derived from a measurement of a medical monitoring device, for example, via an appropriate sensor. In some embodiments, the health related parameter may include such parameters as, but not limited to: breath related parameters (such as, for example, CO₂ related parameters, EtCO₂, O₂ related parameters, breath rate, breath cycle, respiration rate, and the like); heart related parameters (such as, pulse rate); blood related parameters (such as, blood pressure); temperature; skin related parameters (such as, skin humidity, skin conductance, skin capacitance, and the like); Brain related parameters (such as, brain activity); and the like; or combinations thereof.

As referred to herein, the terms “patient” and subject” may interchangeably be used and may relate to a subject being monitored by any monitoring device for any physical-condition related parameter and/or health related parameter. In some embodiments, the subject is capable of responding to instructions provided by a communicating device or system.

As referred to herein, the term “waveform” is directed to a recurring graphic shape which may be realized by measuring a physiological parameter of a subject over time, such as, for example, concentration of CO₂ in breath, follow rate of breath, electrocardiogram (ECG), plethysmograph, and the like. In some embodiments, a waveform is a medically, time resolved waveform. A waveform may have various characteristic parameters/features/factors that may be derived from the shape, dimension, rate or frequency, reoccurrences, and the like, and combinations thereof.

As referred to herein, the terms ordinary, normal, typical, standard and common may interchangeably be used.

As referred to herein, the term “EtCO₂” relates to End tidal CO₂. The CO₂ is exhaled out of the body and the concentration of the exhaled CO₂, also known as end tidal CO₂ (EtCO₂) is an approximate estimation of the alveolar CO₂ pressure and thus of the arterial levels of CO₂. The values of EtCO2 may be measured in units of pressure, such as, for example, mmHg.

As referred to herein, the term “breath cycle” includes the stages of exhalation and inhalation. The breath cycle may be derived from a CO₂ waveform which depicts the change in expired CO₂ Volume over time, (EtCO₂). During a breath cycle, the levels of CO₂ initially increase as a result of CO₂ release from the airways, from what is known as the “dead space”, which is the space in which no gas exchange takes place. Then, the CO₂ rapidly reaches a plateau at high levels of CO₂, which corresponds to the release of CO₂ from the lungs, in the exhalation phase. A rapid decline in exhaled CO₂ proceeds the inhalation phase, characterized by absence/minute levels of CO_{2.}

As referred to herein, the term “Respiration Rate” (RR) may be defined as the number of breaths taken in a minute, and it may change under various physiological and medical conditions.

As referred to herein, the terms “Heart Rate” (HR) and “pulse rate” may interchangeably be used and may relate to the number of heart pulses (beats) in a minute. Pulse rate is usually considered to be a combination of left ventricular stroke volume, ejection velocity, the relative compliance and capacity of the arterial system, and the pressure waves that result from the antegrade flow of blood and reflections of the arterial pressure pulse returning from the peripheral circulation, and some or all of which may be effected by CO₂.

In some embodiments, the terms “calculated” and “computed” may interchangeably be used.

The term “condition” is directed to the physiological (health) condition of the subject, and/or changes to the condition over time. For example, the condition may include respiratory status of the subject and/or change in the respiratory status over time. For example, the respiratory status may include respiration rate or depth. If the respiration rate or respiration rate is determined to reduce, an instruction may be issued to alleviate the condition (for example, the instruction may be: “take a deep breath”).

Capnography is a non-invasive monitoring method used to continuously measure CO₂ concentration in exhaled breath. The CO₂, which is a constant metabolism product of the cells, is exhaled out of the body, and the concentration of the exhaled CO₂, also known as end tidal CO₂ (EtCO₂), is an approximate estimation of the arterial levels of CO₂. Capnograph (or capnometer) is a medical monitoring device that may be used for measuring the carbon dioxide (CO₂) content in inspired and expired air of a subject. It is a non-invasive device that measures the concentrations of respired gases.

Pulse oximeter is a type of oximeter, which is a medical monitoring device that may be used to determine oxygen saturation of the blood. Pulse oximeter may indirectly be used to measure the oxygen (O₂) saturation concentration and changes in blood volume in the skin (i.e., act as a photoplethysmograph). Pulse oximeter may also be used to measure the pulse rate of a subject. The term SpO₂ relates to the saturation of peripheral oxygen. It is a measurement of the amount of oxygen attached to the hemoglobin in red blood cells in the circulatory system. SpO₂ values are generally given as a percentage. SpO₂ may be monitored and measured by a Pulse Oximeter.

According to some embodiments, there are provided methods and systems, devices and methods for communicating with a subject or a health care provider, by providing instructions to the subject that are related to the condition of the subject, based on various physiological parameters of the subject, being monitored by one or more medical monitoring devices.

In some embodiments, the systems, devices and methods provided herein take use of measurements of physiological parameters and/or information/data derived therefrom, obtained from one or more medical monitoring devices. In some embodiments, the medical monitoring devices may be selected from, but not limited to: capnograph, photoplethysmograph (such as, pulse oximeter), ECG, BIS, EEG, and the like. In some embodiments, the physiological parameters, or data derived therefrom, are such parameters as, for example, but not limited to: breath related parameters (as determined, for example, based on expired CO₂ (measurements of CO₂ in exhaled breath), heart related parameters (such as heart rate), blood related parameters (such as, blood pressure, oxygen saturation, blood gases), brain activity related parameters (such as brain electrical activity), body temperature, and the like, or combinations thereof.

According to some embodiments there is provided a system for communicating with a subject, the system may include one or more medical monitoring devices configured to obtain, acquire or measure one or more physiological parameters of a subject; and a communicating device capable of integrating the measured physiological parameters to determine the condition of the subject and to provide instructions to the subject, based on the condition of the subject or changes to the condition of the subject.

In some embodiments, the system may include one monitoring device. In some embodiments, the system may include more than one monitoring device. In some embodiments, the system may include a combination of monitoring devices. In some embodiments, the system comprises a combination of at least three medical monitoring devices. In some embodiments, the medical monitoring devices may be selected from, but not limited to: a capnograph, a pulse oximeter, heart monitor, brain activity monitor, airflow meter, and the like, or any combination thereof. In some embodiments, the communicating device may integrate more than one discrete physiological parameter. In some embodiments, the integrating device may integrate at least two discrete physiological parameters. In some embodiments, the communicating device may integrate at least three discrete physiological parameters. In some embodiments, the measured physiological parameters may include such parameters as, but not limited to: breath related parameters (such as, CO₂ measurements in exhaled breath, airflow, respiration rate, respiration effort, and the like), heart rate related parameters (such as heart rate), blood related parameters (such as, blood pressure, oxygen saturation, venous return flow from peripherals, and the like), brain activity related parameters (such as brain electrical activity), body temperature, parameters derived therefrom, or combinations thereof.

In some embodiments, the measured parameters may be further manipulated and/or processed to generate data related to the measurements, prior to or concomitantly while being integrated by the communicating device. For example, in some embodiments, various CO₂ related parameters may be derived from the measurement of expired CO₂, such as, for example but not limited to: CO₂ waveform and parameters related thereto (such as, for example, but not limited to: EtCO2, changes in EtCO₂, a slope of the increase in the CO₂ concentration, a change in a slope of the increase in the CO₂ concentration, time to rise to a predetermined percentage of a maximum value of CO2 concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), respiration rate, breath cycle, CO₂ concentration in expired air, and the like, or any combination thereof. For example, in some embodiments, various parameters (data) derived from pulse oximetry measurements may be obtained, such as, heart rate (pulse rate), respiration effort, amplitude of cardiac pulses, as determined based on the photoplethysmogram signal; modulation of the amplitude of cardiac pulses, percent modulation (PMod) of the signal, changes in the PMod, change in venous return flow from the peripherals, and the like, or combinations thereof.

In some embodiments, the measured parameters and/or the data related thereto may be used to determine the condition of the subject and provide instructions to the subject and/or to the health care provide that can reduce, alleviate and/or diminish the condition or symptoms associated with the condition. In some embodiments, a combination of measured parameters and/or data related thereto may be used. In some embodiments, the parameters or data related thereto may be obtained from one or more medical monitoring devices.

According to some embodiments, the communicating device may include one or more functional units. In some embodiments, the device may include a processing logic unit (processing unit). The processing unit may include any type of hardware and software. The processing unit may be used to obtain, receive and/or integrate data obtained from one or more medical monitoring devices and to determine, based on the data provided, the condition of the subject being monitored and/or changes to the condition. In some embodiments, the processing logic may further be used to issue instructions to the subject and/or to health care provider. In some embodiments, the instructions are related to the condition of the subject. In some embodiments, the instructions may be selected from a pre-set of instructions that may be stored, for example, in the memory of the processing unit. In some embodiments, the pre-set of instructions may be predetermined, based on known conditions and the matching between a detected/determined condition and the appropriate instructions may be performed by the processing logic. In some embodiments, the matching between the appropriate instructions and the corresponding condition may be achieved by utilizing various algorithms, learning systems and/or based on protocolized procedures. The pre-set of instructions may be predetermined and may be changed by a user.

In some embodiments, the communicating device may further include an input unit, allowing the addition of various sets of instructions, for example, by recording the instructions, typing the instructions, and the like. In some embodiments, the instructions may be stored in the memory of the processing unit.

In some embodiments, the communicating device may further include a presenting unit that may include an audio presenting unit, a visual presenting unit, or both.

According to some embodiments, the audio presenting unit of the communicating device may include any audio unit, such as, a speaker, earphone, and the like that is capable of providing/emitting sound. In some embodiments, the audio presenting unit may be used to provide audible instructions to the subject. In some embodiments, the audio presenting unit may be used to provide audible instructions to the health care provider. In some embodiments, the audio presenting unit may be controlled by a user that may determine the volume, sound, speech, language, and the like, of the instructions.

According to some embodiments, the visual presenting unit of the communicating device may include any visual unit, such as, a monitor, display, screen, and the like that is capable of presenting instructions. In some embodiments, the visual presenting unit may be used to provide visual instructions to the subject, for example, in the form of written instructions. In some embodiments, the visual presenting unit may be used to provide visual instructions to the health care provider. In some embodiments, the visual presenting unit may be controlled by a user that may determine the size, type, brightness, and the like, of the instructions presented to the subject and/or the health care provider. In some embodiments, the visual presenting unit is a bed-side monitor.

According to some embodiments, the measured physiological parameters may be determined or calculated over a period of time, in order to effectively determine the condition of the subject over time. The period of time may be predetermined.

According to some embodiments, the communicating device may further include a user interface that may allow a user to select various operating parameters. In some embodiments, the user interface may be used by the subject and/or the health care provider to input/provide instructions and/or feedback to the device. For example, the user interface may be operated by the subject and/or health care provider by pressing a key, pressing a button, typing on a keyboard, talking to a microphone of the device, and the like. In some embodiments, the feedback/instructions that may be provided/input to the device may include such inputs as, but not limited to: “confirm” status, “start” a procedure, “start” operation of the device, “end” a procedure, “end” operation of the device, “cancel” a procedure, “cancel” operation of the device, “confirm” the instructions from the device are met by the subject and/or health care provider, and the like.

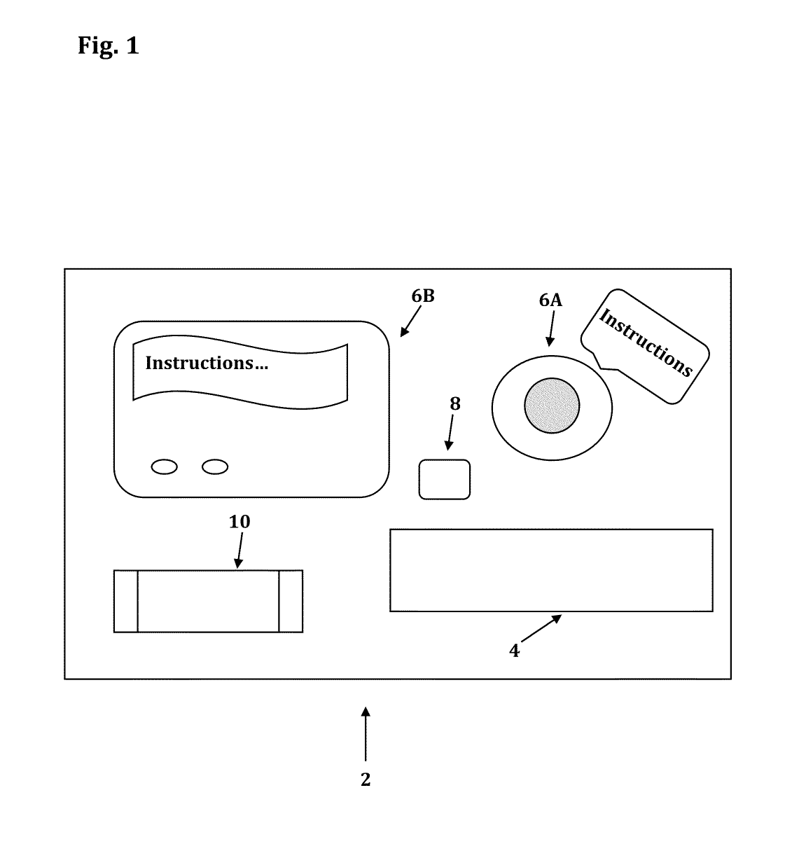

Reference is now made to FIG. 1, which is a schematic block diagram of a communicating device, according to some embodiments. As shown in FIG. 1, the device (2) may include a processing unit, (4), capable of obtaining, receiving information/data from one or more medical monitoring devices and determine the condition of the subject, based on the data. The processing unit may further determine if instructions are to be provided to the subject and which instructions are to be provided. The processing unit may further activate/control a presenting unit configured to provide/present the instructions to the subject. The processing unit may include any type of firmware, hardware and/or software. The device may further include a presenting unit that may include an audio presenting unit (6A, shown as a speaker) and/or a visual presenting unit (6B, shown as a monitor). The presenting unit is configured to provide/present instructions to the subject. The instructions may be provided, for example, in the form of audible instructions (for example, verbal instructions provided by a speaker, as illustrated in FIG. 1), and/or in the form of visual instructions (for example, written instructions, presented on a monitor, as shown in FIG. 1). The device may further include an input unit (8), configured to allow a user to input/add various instructions. The input unit may include, for example, a voice recorder, configured to record instructions that may be stored in the device (for example, in the memory of the processing unit). The device may further include a user interface that may include any type of interface allowing a user to control various operating parameters of the device and/or provide feedback or other operating instructions from the user to the device. For example, in FIG. 1, the user interface is shown in the form of keyboard (10).

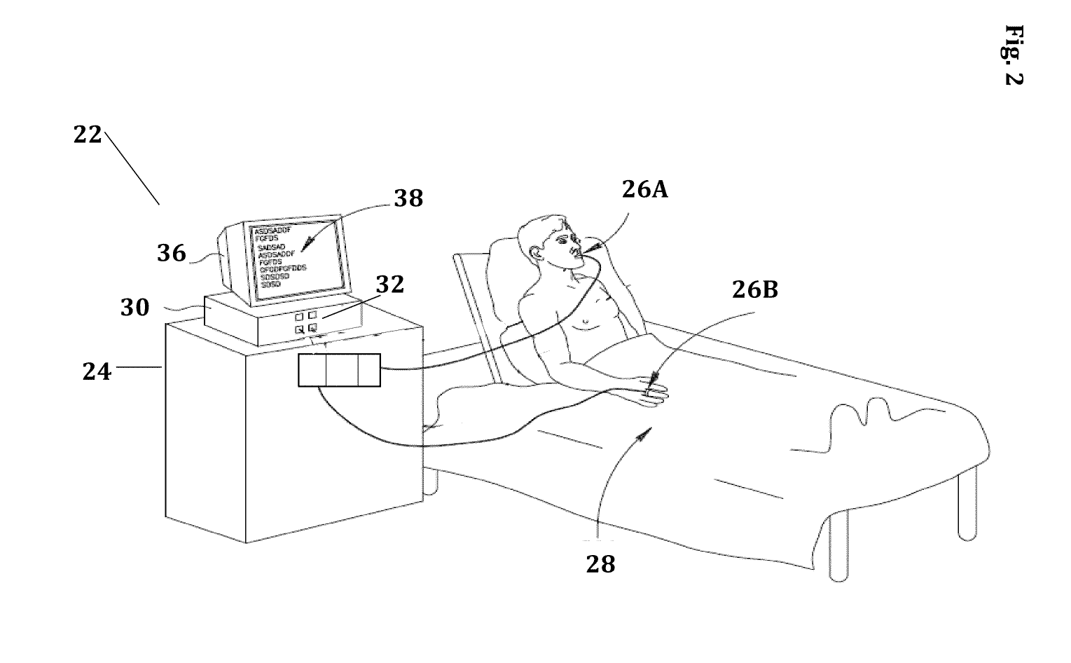

Reference is now made to FIG. 2, which is a schematic illustration of a system, according to some embodiments. As shown in FIG. 2, system (22) may include one or more medical monitoring devices (shown as one integrated device (24)). The medical monitoring devices may include, for example, but not limited to: a capnograph, a pulse oximeter, spirometer, heart rate sensors, blood pressure sensors, ECG, EEG, and the like. The monitoring devices include various sensors, such as, sensors 26A-B that may be configured to obtain/sense/measure various physiological parameters of subject (28). The exemplary sensors shown in FIG. 2 are CO₂ sensor (26A) and pulse oximetry sensor (26B). The one or more sensors may be connected directly or indirectly to the subject. The parameters thus measured/obtained/calculated may include, for example, such parameters as, but not limited to: EtCO₂, CO₂ levels, CO₂ waveform pattern, SpO₂, heart rate, blood pressure, blood flow, blood gases, and the like. System 22 may further include a communicating device, (30), that may be used to receive information from the medical monitoring devices, to process the information and determine the condition of the subject, based on the measured parameters or data derived therefrom. The communicating device may further issue/provide instructions to the subject and/or a health care provider, based on the condition of the subject. The connection between the communicating device and the medical monitoring device (or any sensor(s) thereof) may include any type of communication route, such as, for example, use of wires, cables, wireless, and the like. The communicating device may include a signaling unit, such as, audio signaling unit (32), configured to provide audible instructions to the subject. The communication device may further include a visual signaling unit (shown, for example, as monitor 36 in FIG. 1), that may be used to present the instructions (38) to the subject. The communicating device may further include data storing subunits, capable of storing data, such as, sets of instructions, historical data, subject related data, measured parameters of data derived therefrom, and the like.

According to some embodiments, the instructions provided by the device and system disclosed herein may be provided to the subject and/or the health care provider. The instructions provided may include such instructions as, but not limited to: instructions for an intervention procedure to be taken by the subject, instructions for an intervention procedure to be taken by the health care provider; an indication to the subject, an information provided to the subject, and the like, or combinations thereof. In some embodiments, the instructions are verbal instructions. In some embodiments, the instructions are messages. In some embodiments, the instructions may be provided/presented in the form of audible instructions, visual instructions, or both.

According to some embodiments, the instructions may include information to the subject regarding his healthcare. For example, the instructions provided may inform the subject that a health care provider is on his way to attend to the subject. In some embodiments, the instructions may also provide an estimation of the time of arrival of the health care provider. Such instructions may be provided based on the medical condition of the subject and/or may be provided/input to the system by a health care provider.

According to some embodiments, the device and system disclosed herein may relay information of the health condition (status) of the subject, to the subject and/or to the health care provider, thereby informing of improvement or deterioration in the condition. In some embodiments, the instructions may further include recommendations or suggestions for means of improving the health condition of the subject.

According to some embodiments, the device and systems disclosed herein may provide/issue a series of verbal command to the subject as part of a protocolized procedure. For example, when testing or monitoring breathing of a subject, the device may issue instructions, (for example, in the form of commands), related to breathing. For example, in some embodiments, the device may issue breathing commands so that the subject breaths in a regular pattern, for determination of breathing effort, or for baseline value of other related parameters, such as, for example, but not limited to: heart rate, percent modulation of pleth (pMod), and the like.

According to some embodiments, the device and system disclosed herein may provide/issue instructions to a healthcare provider attending the subject. For example, in some embodiments, such instructions may include instructions advising/reminding the health care provider that a drug is to be administered.

According to some embodiments, the device and system disclosed herein may provide/issue instructions which include a series of commands within a protocolized procedure. For example, in some exemplary embodiments, during a passive leg raising (PLR) maneuver that is used for the determination of fluid responsiveness, the device disclosed herein may issue commands for the subject to be moved to each position during the maneuver. The instructions provided may further include advising the subject during the procedure to relax and breath regularly, and the like, in order to improve the repeatability and effectiveness of the procedure and to reduce work load of the health care provider. The instructions may include a pre-set, predetermined set of instructions. The instructions provided is such a setting may be initiated by the health care provider and/or based on parameters obtained from the one or more monitoring device.

According to some embodiments, the device and system disclosed herein may be used for monitoring breathing of semi-sedated subjects and/or subjects recovering from surgery which are treated by the pain killers that can also affect breathing. In such settings, the device and system, upon detection of reduced breath rate or reduced breath depth may provide instructions to the subject to take a deep breath. The instructions provided by the device and system disclosed herein may be used to replace instructions that are usually provided by a health care provider, such as a nurse. In some embodiments, the device and system may further provide instructions to the health care provider, if the intervention instructions provided to the subject did not result in improvement of the condition, or if the condition persists.

According to some embodiments, there is provided a method for communicating with a subject, the method comprising one or more of the steps of:
a. obtaining a measurement of one or more physiological parameters of the subject;
b. determining the condition of the subject based on the measured parameters of the subject; and
c. issuing audible and/or visual instructions to the subject, wherein the instructions are related to the condition of the subject and may be used to alleviate said condition or to inform the subject of his condition.

In some embodiments, the physiological parameters of the subject comprises: breath related parameters, heart related parameters, blood related parameters, brain electrical activity, parameters derived therefrom, or combinations thereof.

According to some embodiments, the instructions may be selected from a predetermined set of instructions. In some embodiments, the instructions may be provided automatically, as determined based on the condition of the subject. In some embodiments, the instructions may be provided upon indication by a health care provider.

According to some embodiments, the instructions comprises instructions for an intervention procedure to be taken by the subject, instructions for an intervention procedure to be taken by the health care provider; an indication to the subject, or combinations thereof.

In some embodiments, the method may further include manipulation and/or processing of the measured parameters, to generate data related to the measurements, prior to or simultaneously while being integrated for determination of the condition of the subject. In some embodiments, the measured physiological parameters may be determined or calculated over a period of time, in order to effectively determine the condition or changes thereto. The period of time may be predetermined.

According to some embodiments, learning and optimization of determining a condition of a subject may be carried out using various methods, such as, for example, but not limited to: neural networks, a support vector machine (SVM), genetic algorithms, simulated annealing and expectation-maximization (EM), learning systems based on historic data, and the like.

In some exemplary embodiments, various CO₂ related parameters may be derived from the measurement of CO₂ in breath, such as, for example but not limited to: CO₂ waveform and parameters related thereto (such as, for example, but not limited to: EtCO2, changes in EtCO₂, a slope of the increase in the CO₂ concentration, a change in a slope of the increase in the CO₂ concentration, time to rise to a predetermined percentage of a maximum value of CO2 concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), respiration rate, breath cycle, CO₂ concentration in expired air, and the like, or any combination thereof.

For example, in some embodiments, various parameters (data) derived directly or indirectly from pulse oximetry measurements may be obtained, such as, heart rate (HR, pulse rate), respiration effort (RE), amplitude of cardiac pulses (as determined based on the photoplethysmograph signal (PPG)); modulation of the amplitude of cardiac pulses, percent modulation (PMod) of the signal, changes in the PMod, changes in blood flow (for example, change in venous return flow from the peripherals), and the like, or combinations thereof.

According to some embodiments, there is provided a method used in a system for communicating with a subject, the system may include one or more medical monitoring devices configured to measure one or more physiological parameters of a subject; a communicating device configured to integrate the one or more physiological parameters of the subject to determine the condition of the subject, based on the measured parameters; and issue/provide instructions to the subject or to a health care provider, wherein the instructions are related to the condition of the subject. According to some embodiments, the physiological parameters may be such parameters as, but not limited to: expired CO2, CO2 waveforms, airflow, respiration rate, respiration effort, heart rate, SpO₂, blood pressure, brain activity, data derived therefrom or related the.

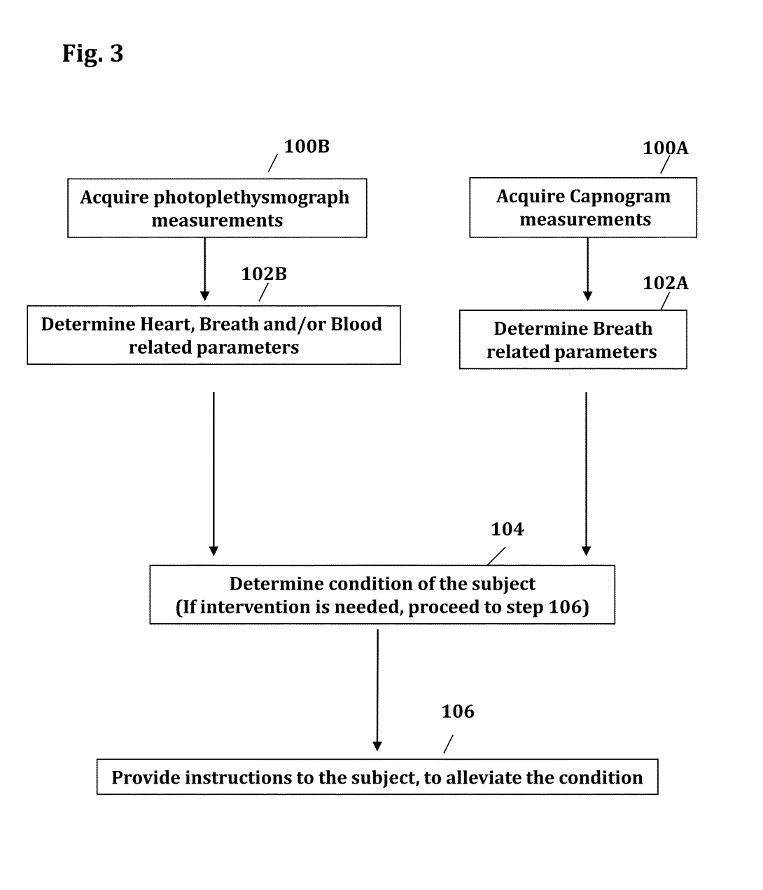

Reference is now made to FIG. 3, which is a schematic block diagram of steps in a method for communicating with a subject, according to some embodiments. As shown in FIG. 3, measurements of one or more physiological parameters of a subject are obtained from the appropriate corresponding monitoring devices. In the example shown in FIG. 3, at step 100A, capnograph measurements are obtained, and at step 100B, photoplethysmograph (pulse oximetry) measurements are obtained. Each of steps 100A-B may be performed simultaneously, sequentially or independently of each other. Next, the obtained measurements may be further proceed or analyzed to determine various related parameters. For example, at step 102A, various breath related parameters (such as, for example, airflow, respiration rate, expired CO₂ related parameters) are determined, based on the capnograph measurements. For example, at step 102B, various breath, heart and/or blood related parameters are determined, based on the pulse oximetry measurements. Such exemplary parameters may be such parameters as, but not limited to: heart rate (pulse rate), respiration rate, respiration effort, amplitude of cardiac pulses; modulation of the amplitude of cardiac pulses, percent modulation (PMod) of the signal, vasoconstriction, changes in venous blood return from peripheries, and the like, and combinations thereof. Each of steps 102A-B may be performed simultaneously, sequentially or independently of each other. Next, at step 104, the various parameters that have been determined in steps 102A-B may be integrated (for example, by a communicating unit) to determine the condition of the subject. Further in step 104, if based on the provided measurements (or data derived therefrom) an intervention procedure is needed, instruction are provided in step 106. The instructions provided in step 106 may be provided in the form of audible instructions and/or visual instructions. For example, the instructions may include verbal instructions. The instructions provided in step 106 may be configured to alleviate the condition. For example if a shallow breathing is detected, the instructions provided may include verbal instructions to the subject “take a deep breath”.

According to some embodiments, the devices, systems and methods disclosed herein provide for an automatic or semi-automatic communication with a subject, based on the condition of the subject.

It is understood by the skilled in the art that the processor of the system is configured to implement the method as essentially described herein.

In the description and claims of the application, each of the words “comprise” “include” and “have”, and forms thereof, are not necessarily limited to members in a list with which the words may be associated.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

### EXAMPLES

### Example 1

### Communicating with a Subject—Performing Passive Leg Raising (PLR) Maneuver

Passive leg raising (PLR) test is a bedside test to evaluate the need for further fluid resuscitation in ill subjects. The test involves raising the legs of a patient, which causes gravity to pull blood from the legs, thus increasing circulatory volume available to the heart. The real-time effects of this maneuver on hemodynamic parameters, such as blood pressure and heart rate are used to guide the decision whether or not more fluid will be beneficial. The device and system disclosed herein are used to provide a full set of instructions to the subject in performing the maneuver.

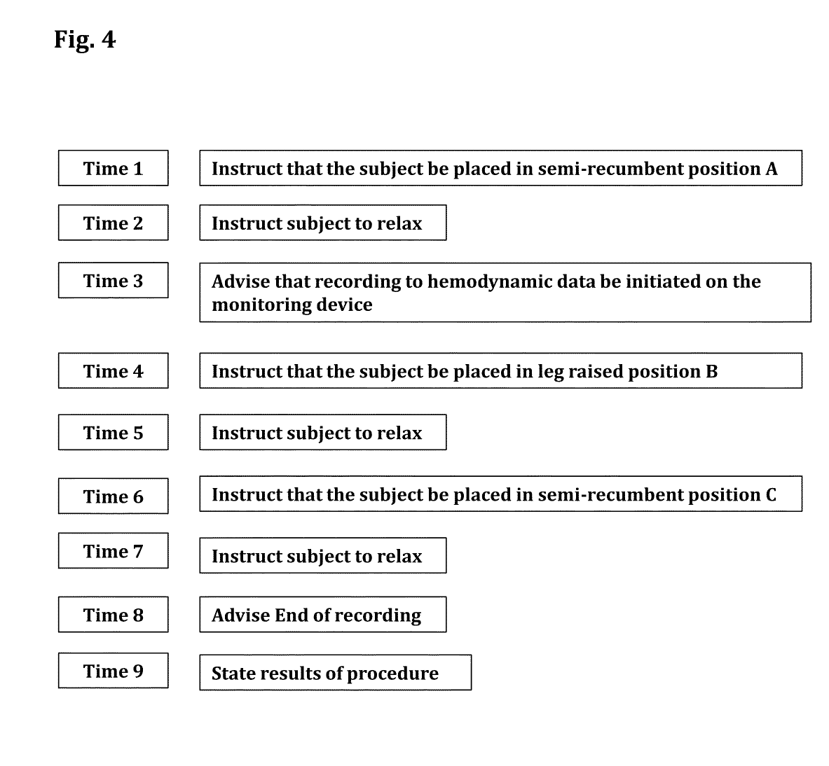

Shown in FIG. 4 is an exemplary set of instructions provided by the device and system to the subject for performing the PLR maneuver. At each time point, an appropriate command is provided, based on the condition of the subject. The use of the device and system provided herein allows for automatic execution of the maneuver and measurements thereof (i.e., without intervention of a health care provider to provide instructions and/or perform measurements).

The examples described above are non-limiting examples and are not intended to limit the scope of the disclosure. The described examples may comprise different features, not all of which are required in all embodiments of the disclosure.

What is claimed is:

1. A system for communicating with a subject, the system comprising:
one or more medical monitoring devices configured to measure one or more physiological parameters of the subject;
a communicating device configured to integrate the one or more physiological parameters of the subject to determine the condition of the subject and/or changes to the condition of the subject, based on the measured parameters and provide instructions to the subject or to a health care provider, wherein the instructions are related to the condition of the subject.

2. The system of claim 1, wherein the medical monitoring device comprises: capnograph, pulse oximeter, Electrocardiogram (ECG), Brain activity monitoring device, or combinations thereof.

3. The system of claim 1, wherein the physiological parameters of the subject comprises: breath related parameters, heart related parameters, blood related parameters, brain electrical activity, parameters derived therefrom, or combinations thereof.

4. The system of claim 1, wherein the instructions comprise visual and/or audible instructions.

5. The system of claim 1, wherein the instructions comprise instructions for an intervention procedure to be taken by the subject, instructions for an intervention procedure to be taken by the health care provider; an indication to the subject, or combinations thereof.

6. The system of claim 1, wherein the communicating device comprises a display unit and/or an audio unit.

7. The system of claim 1, wherein the instructions are selected from a predetermined set of instructions.

8. The system of claim 7, wherein the display unit is configured to display one or more of: one or more of the measured parameters, parameters derived from the measured parameters, an indication for an intervention to be taken by the subject, an indication for an intervention to be taken by a health care provider, an indication related to a health care provider, or combinations thereof.

9. The system of claim 7, wherein the audio unit comprises a speaker, said audio unit is configured to provide audible instructions to the subject and/or to the health care provider.

10. The system of claim 7, wherein the communicating device further comprises a processing logic.

11. A device for communicating with a subject, the device comprising:
a) a processing logic configured to obtain and integrate one or more measured physiological parameters of the subject, obtained from one or more medical monitoring devices, to determine the condition of the subject, and
b) a display unit and/or an audio unit configured to provide instructions to the subject and/or to a health care provider, wherein the instructions are related to the condition of the subject.

12. The device of claim 11, wherein the medical monitoring device comprises: a capnograph, pulse oximeter, Electrocardiogram (ECG), Brain activity monitoring device, or combinations thereof.

13. The device of claim 11, wherein the instructions are selected from a predetermined set of instructions, said instructions comprises instructions for an intervention procedure to be taken by the subject, instructions for an intervention procedure to be taken by the health care provider; an indication to the subject, or combinations thereof.

14. The device of claim 11, wherein the display unit comprises a monitor.

15. The device of claim 11, wherein the audio unit comprises a speaker.

16. The device of claim 11, further comprising a user interface configured to control operating parameters of the device, said operating parameters comprises: volume, type of voice providing instructions, operating language, language of voice providing instructions, a recorder configured to pre-record instructions, or combinations thereof.

17. A method for communicating with a subject, the method comprising:
a. obtaining a measurement of one or more physiological parameters of the subject;
b. determining the condition of the subject based on the measured parameters of the subject; and
c. issuing audible and/or visual instructions to the subject, wherein the instructions are related to the condition of the subject and are configured to alleviate said condition.

18. The method of claim 17, wherein the physiological parameters of the subject comprises: breath related parameters, heart related parameters, blood related parameters, brain electrical activity, parameters derived therefrom, or combinations thereof.

19. The method of 17, wherein the instructions are selected from a predetermined set of instructions.

20. The method of claim 17, wherein the instructions comprises instructions for an intervention procedure to be taken by the subject, instructions for an intervention procedure to be taken by the health care provider; an indication to the subject, or combinations thereof.
